(19) United States
(12) Patent Application Publication
(10) Pub. No.: US 2015/0311453 A1
(43) Pub. Date: Oct. 29, 2015

(54) ORGANIC ELECTROLUMINESCENCE ELEMENT AND ILLUMINATION DEVICE

(71) Applicant: PANASONIC INTELLECTUAL PROPERTY MANAGEMENT CO., LTD., Osaka (JP)

(72) Inventors: Hiroya TSUJI, Kyoto (JP); Satoshi OHARA, Tokyo (JP); Nobuhiro IDE, Osaka (JP)

(73) Assignee: PANASONIC INTELLECTUAL PROPERTY MANAGEMENT CO., LTD., Osaka (JP)

(21) Appl. No.: 14/647,918

(22) PCT Filed: Nov. 12, 2013
(86) PCT No.: PCT/JP2013/006647

§ 371 Date: May 28, 2015

(30) Foreign Application Priority Data
Nov. 30, 2012 (JP) 2012-263281

(51) Int. Cl. H01L 51/00

(52) U.S. Cl. CPC H01L 51/0072; H01L 51/0067; H01L 51/007; H01L 51/0052; H01L 51/5278

(57) ABSTRACT

The organic electroluminescence element includes: a positive electrode; a negative electrode; a plurality of light emitting layers interposed between the positive electrode and the negative electrode; and an interlayer-provided between the plurality of light emitting layers. The interlayer includes: a first layer-containing a nitrogen-containing heterocyclic compound; an alkali metal layer containing an alkali metal; a second layer containing a nitrogen-containing heterocyclic compound; and a hole injection layer containing an electron-accepting organic material. The first layer, the alkali metal layer, the second layer, and the hole injection layer are arranged in this order from the positive electrode to the negative electrode. The second layer is thicker than the alkali metal layer.

### TECHNICAL FIELD

The present invention relates to an organic electroluminescence element available for illuminating light sources, backlights for liquid crystal displays, and flat panel displays or the like, and an illumination device including the organic electroluminescence element.

### BACKGROUND ART

As an example of organic light emitting elements referred to as organic electroluminescence elements, there is known an element which includes a transparent electrode serving as a positive electrode, a hole transporting layer, a light emitting layer (organic light emitting layer), an electron injection layer, and an electrode serving as a negative electrode stacked in this order on a surface of a transparent substrate. When a voltage is applied between the positive and negative electrodes, electrons injected into the light emitting layer via the electron injection layer and holes injected into the light emitting layer via the hole transporting layer recombine with each other in the light emitting layer, and thereby light is generated. The light generated in the light emitting layer is allowed to emerge outside through the transparent electrode and the transparent substrate.

The organic electroluminescence element is characterized in that the element is self-luminous, shows relatively highly-efficient emission characteristics, and allows emission of light in various color tones. Specifically, it is expected to use the organic electroluminescence element as light emitters in display devices such as flat panel displays, or as light sources such as backlights for liquid crystal displays and illumination. Some of the organic electroluminescence elements are already put to practical use.

The organic electroluminescence element has a trade-off between the luminance and the lifetime. Therefore, there has been actively developed an organic electroluminescence element which retains the lifetime even when the luminance of light is increased to obtain a more clear image or brighter illuminating light.

Specifically, there have been proposed organic electroluminescence elements in which a plurality of light emitting layers are interposed between a positive electrode and a negative electrode and the light emitting layers are electrically connected (see, for example, Patent Literatures 1 to 6).

FIG. 3 shows an example of the structure of the organic electroluminescence element. A plurality of light emitting layers 4 and 5 are provided between an electrode serving as a positive electrode 1 and an electrode serving as a negative electrode 2. The plurality of light emitting layers 4 and 5 are stacked in a state where an interlayer 3 is interposed between the adjacent light emitting layers 4 and 5. This structure is disposed on a surface of a transparent substrate 10. For example, the positive electrode 1 is formed as a light-transmitting electrode, and the negative electrode 2 is formed as a light-reflecting electrode. An electron injection layer and a hole transporting layer provided on both sides of the light emitting layers 4 and 5 are not shown in FIG. 3.

In the configuration, the interlayer 3 is interposed between the plurality of light emitting layers 4 and 5 so as to electrically connect them to each other. When a voltage is applied between the positive electrode 1 and the negative electrode 2, the plurality of light emitting layers 4 and 5 emit light simultaneously as if the light emitting layers 4 and 5 are connected in series with each other. In this case, rays of light emitted from the light emitting layers 4 and 5 are combined. Thereby, when a constant current is supplied, luminance in the organic electroluminescence element is higher than luminance in a conventional organic electroluminescence element. Thus, the problem of the trade-off between the luminance and the lifetime is solved.

Herein, examples of known common configurations of the interlayer 3 include (1) BCP:Cs/V₂O₅, (2) BCP:Cs/NPD:V₂O₅, (3) in-situ reaction products of a Li complex and Al, (4) Alq:Li/ITO/hole transporting materials, (5) mixed metal-organic layers, (6) oxides containing alkali metals and alkali-earth metals, (7) N-doped layer/metal oxide layer/P-doped layer. The character “:” means a mixture of two materials, and the character “/” means a layered structure of the former and latter compositions.

### CITATION LIST

### Patent Literature

Patent Literature 1: JP 2003-272860 A

Patent Literature 2: JP 2005-135600 A

Patent Literature 3: JP 2006-332048 A

Patent Literature 4: JP 2006-173550 A

Patent Literature 5: JP 2006-49393 A

Patent Literature 6: JP 2004-281371 A

### SUMMARY OF INVENTION

### Technical Problem

However, the above organic electroluminescence element may cause an increase in an operation voltage and an undesired increase in a voltage, and also may disadvantageously cause defects such as short circuit due to poor film quality. In particular, in a high temperature environment, the organic electroluminescence element is apt to cause the increase in an operation voltage, and may cause large deteriorations in performance and quality depending on a temperature environment.

Specifically in the interlayer of the system shown in the item (1), the defects may be disadvantageously caused by short circuit due to the poor quality of the V₂O₅ layer.

In the system shown in the item (2), an increase in a voltage may be disadvantageously caused by the side reaction between the two layers. That is, it is reported that Lewis acid molecules react with the electron-transporting material and alkali metals react with the hole transporting material as Lewis bases, to cause an increase in an operation voltage (reference literature: Multiphoton Organic EL Illumination, Meeting of Research Group on Organic EL Material & Device, The Society of Polymer Science, on Dec. 9, 2005).

In the system shown in the item (3), the organic ligand component of a Li complex used for obtaining an in-situ reaction product may disadvantageously exert adverse effects on the element characteristics.

In the system of the item (4), hole injection from ITO as the interlayer to the hole transporting material is not always favorable. This may cause problems on an operation voltage and element characteristics. Furthermore, since the specific resistance of ITO is small, electric charges may migrate on the ITO surface to the places where no emission is essentially desired, which may disadvantageously cause light emission from regions other than the intended light emission region.

In the system of the item (5), the interlayer is formed by mixing an organic matter with metal including a metal compound such as metal oxide, which may cause deterioration in the thermal stability of the interlayer, and particularly cause degradation of the interlayer by heat generation resulting from a flow of a large current.

In the system of the item (6), the function of the interlayer of a metal oxide containing an alkali or alkali-earth metal is not necessarily sufficient. Therefore, it is substantially necessary to use a layer made of a substance other than the metal oxide containing an alkali or alkali-earth metal in a state where the layer is disposed, which leads the complicated structure of the interlayer and causes problems on the production of the layer.

In the system of the item (7), it is disclosed that a layer for preventing diffusion of a metal oxide is provided in order to prevent mutual diffusion of the P and N dopants in the interlayer. However, from the viewpoint of OLED design, provision of a layer made only of a metal oxide as the interlayer is equivalent to provision of a component having a refractive index higher than that of the principal organic matter in the OLED. In this case, a large refractive index difference (0.2 or more) occurs in the intermediate part, which tends to cause an increase in optical interference due to the refractive index difference from the optical viewpoint, and causes an increase in a degree of difficulty of optical design. Accordingly, the system is not desirable from the point of emission characteristics such as emission efficiency.

Particularly, the systems of the items (1) to (7) are apt to further cause an increase in an operation voltage and short circuit in a high temperature environment, which cause various problems on durability, lifetime, and the like.

In view of the above problems, an object of the present invention is to provide an organic electroluminescence element and an illumination device which have an improved interlayer and therefore are less likely to cause an increase in an operation voltage and short circuit not only in a room temperature environment but also in a high temperature environment and have excellent long-term durability and lifetime characteristics. Another object of the present invention is to provide an illumination device.

### Solution to Problem

An organic electroluminescence element according to the present invention includes: a positive electrode; a negative electrode; a plurality of light emitting layers interposed between the positive electrode and the negative electrode; and an interlayer provided between two adjacent light emitting layers of the plurality of light emitting layers. The interlayer includes: a first layer containing a nitrogen-containing heterocyclic compound; an alkali metal layer containing an alkali metal; a second layer containing a nitrogen-containing heterocyclic compound; and a hole injection layer containing an electron-accepting organic material. The first layer, the alkali metal layer, the second layer, and the hole injection layer are arranged in this order from the positive electrode to the negative electrode. The second layer is thicker than the alkali metal layer.

In the organic electroluminescence element, the second layer preferably has a thickness in a range of 0.2 to 20 nm.

In the organic electroluminescence element, the nitrogen-containing heterocyclic compound preferably has two or more 1,10-phenanthroline sites or two or more 2,2′-bipyridine sites per molecule.

In the organic electroluminescence element, the nitrogen-containing heterocyclic compound contained in the first layer is preferably the same as the nitrogen-containing heterocyclic compound contained in the second layer.

In the organic electroluminescence element, the electron-accepting organic material is preferably 1,4,5,8,9,11-hexaazatriphenylene-hexacarbonitrile.

An illumination device according to the present invention includes the organic electroluminescence element.

### Advantageous Effects of Invention

The present invention can provide an organic electroluminescence element which includes an interlayer including a specific layer and thereby is less likely to cause an increase in an operation voltage and short circuit not only in a room temperature environment but also in a high temperature environment and has excellent long-term durability and lifetime characteristics.

### BRIEF DESCRIPTION OF DRAWINGS

FIG. 1 relates to an example of an embodiment of the present invention, and is a schematic cross-sectional view showing a configuration of an interlayer of an organic electroluminescence element;

FIG. 2 relates to the example of the embodiment of the present invention, and is a schematic cross-sectional view showing a layer configuration of an organic electroluminescence element; and

FIG. 3 is a schematic cross-sectional view showing a layer configuration of an example of a conventional organic electroluminescence element.

### DESCRIPTION OF EMBODIMENTS

Hereinafter, one embodiment of the present invention will be described.

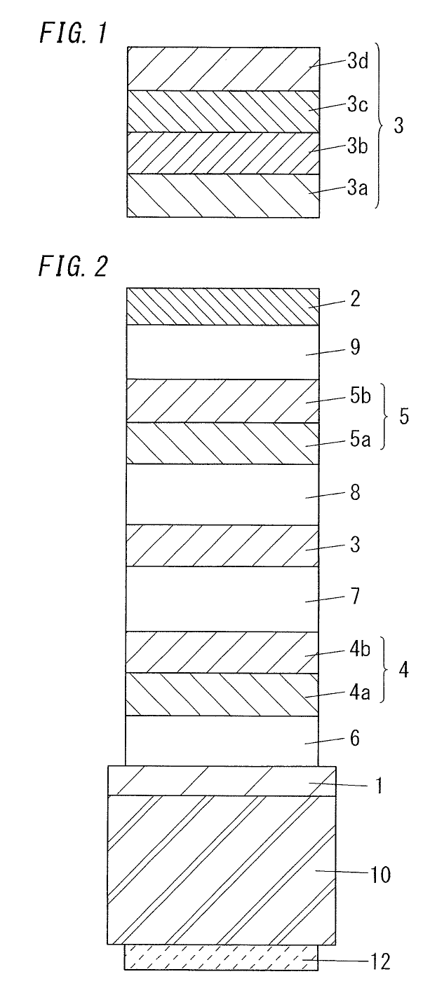

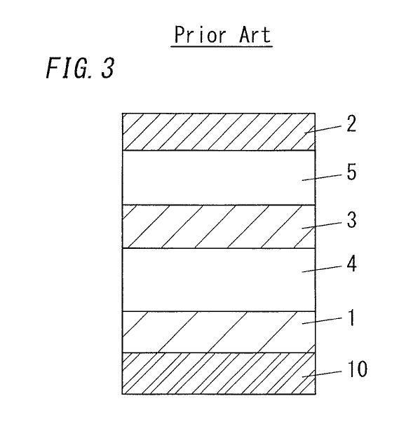

FIG. 1 is a schematic cross-sectional view showing a configuration of an interlayer 3 of an organic electroluminescence element (hereafter, may be referred to as an “organic EL element”) of the present invention. In FIG. 1, configurations of members other than the interlayer 3 are not shown.

FIG. 2 shows an example of the organic electroluminescence element of the present invention of the embodiment, and is a schematic cross-sectional view showing the layer configuration of the organic electroluminescence element. In the organic electroluminescence element of the embodiment, at least a plurality of light emitting layers 4 and 5 and an interlayer 3 are provided between an electrode serving as a positive electrode 1 and an electrode serving as a negative electrode 2. The organic electroluminescence element has a so-called multi-unit structure. The interlayer 3 is disposed between the plurality of light emitting layers 4 and 5. In the present embodiment, the interlayer 3 functions to electrically connect two light emitting units (light emitting layers 4 and 5) in series with each other.

As shown in FIG. 1, the interlayer 3 includes a first layer 3a, an alkali metal layer 3b, a second layer 3c, and a hole injection layer 3d. Although the positive electrode 1 and the negative electrode 2 are not shown in FIG. 1, the interlayer 3 is a layer in which the first layer 3a, the alkali metal layer 3b, the second layer 3c, and the hole injection layer 3d are stacked in this order from the positive electrode 1 to the negative electrode 2. That is, in FIG. 1, the first layer 3a is located closer to the positive electrode 1, and the hole injection layer 3d is located closer to the negative electrode 2.

The alkali metal layer 3b is made of only alkali metal. Examples of alkali metal constituting the alkali metal layer 3b may include Li, K, Na, Cs, Rb, and Fr. The alkali metal layer 3b may be made of any one of the above alkali metals, or be made of two or more of the above alkali metals. Since alkali metal has an electron-releasing property, the alkali metal layer 3b functions to inject electrons.

The thickness of the alkali metal layer 3b is not particularly limited, but is preferably 0.01 to 10 nm. When the thickness of the alkali metal layer 3b is within the above range, it is possible to prevent occurrence of an increase in an operation voltage of the organic EL element, and particularly to reduce an increase in the operation voltage even in a high temperature environment. Thus, the alkali metal layer 3b can function sufficiently. The thickness of the alkali metal layer 3b is more preferably 0.1 to 5 nm.

The first layer 3a is made of a material containing a nitrogen-containing heterocyclic compound, and is formed on a surface of the alkali metal layer 3b being closer to the positive electrode 1.

The nitrogen-containing heterocyclic compound contained in the first layer 3a is a heterocyclic compound (may also be a heterocyclic type compound or a hetero ring type compound) including nitrogen atoms as its constituent atoms. The heterocyclic compound means a cyclic compound containing two or more kinds of elements.

Examples of the nitrogen-containing heterocyclic compound include a 1,10-phenanthroline derivative. For example, a compound having two or more 1,10-phenanthroline sites per molecule can be used. Examples of the compound include a compound represented by the general formula (1) in the following [Chemical Formula 1].

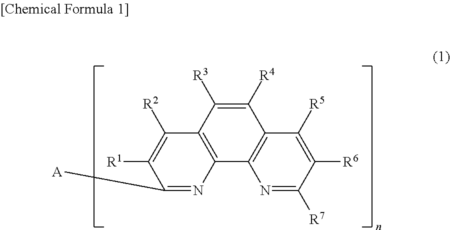

(In the formula, “R¹” to “R⁷” are selected from the group consisting of a hydrogen atom, a hydrocarbon group having 1-10 carbon atoms, and a substituted or unsubstituted aryl group having 6-30 carbon atoms. “A” is a hydrocarbon group having 1-10 carbon atoms, a substituted or unsubstituted aryl group having 6-30 carbon atoms, or a di- or higher-valent aromatic hydrocarbon group having 6-30 carbon atoms. “n” is an integer greater than or equal to 2. “R¹” to “R⁷” may be the same or different.) Herein, when each of “R¹” to “R⁷” is the hydrogen atom in the compound represented by the general formula (1), the compound can be said to have two or more 1,10-phenanthryl groups.

In the general formula (1), examples of the hydrocarbon group having 1-10 carbon atoms include an alkyl group having 1-10 carbon atoms. Specific examples of the alkyl group having 1-10 carbon atoms include methyl, ethyl, n-propyl, isopropyl, n-butyl, 2-butyl, tert-butyl, n-pentyl, 2-pentyl, 3-pentyl, neopentyl, n-hexyl, 2-hexyl, 2-ethylhexyl, 2-butylhexyl, n-heptyl, n-octyl, 2-octyl, n-nonyl, and n-decyl groups. Other examples of the hydrocarbon group having 1-10 carbon atoms include an alkylene group having 1-10 carbon atoms. The hydrogen atom of the hydrocarbon group having 1-10 carbon atoms may be substituted with another functional group (for example, a hydroxyl group or the like).

In the general formula (1), examples of the substituted or unsubstituted aryl group having 6-30 carbon atoms include phenyl, 1-naphthyl, 2-naphthyl, 4-phenyl-1-naphthyl, 1-anthryl, 2-anthryl, 9-anthryl, 10-phenyl-9-anthryl, 1-phenanthryl, 2-phenanthryl, 3-phenanthryl, 4-phenanthryl, 9-phenanthryl, 1-pyrenyl, 2-pyrenyl, 2-perylenyl, 3-perylenyl, 1-fluoranthenyl, 2-fluaranthenyl, 3-fluoranthenyl, 8-fluoranthenyl, 2-triphenylenyl, 9,9-dimethylfluorene-2-yl, 9,9-dibutylfluorene-2-yl, 9,9-dihexylfluorene-2-yl, 9,9-dioctylfluorene-2-yl, 9,9-diphenylfluorene-2-yl, 2-biphenylyl, 3-biphenylyl, 4-biphenylyl, p-terphenyl-3-yl, p-terphenyl-4-yl, m-terphenyl-3-yl, m-terphenyl-4-yl, o-terphenyl-3-yl, o-terphenyl-4-yl, 4-(1-naphthyl)-1-naphthyl, o-tolyl, m-tolyl, p-tolyl, 4-tert-butylphenyl, 4-methyl-1-naphthyl, 4-phenyl-1-naphthyl, 10-methyl-9-anthryl, and 4-phenyl-8-fluoranthenyl groups.

Examples of a substituent group in the case of the substituted or unsubstituted aryl group having 6-30 carbon atoms in the general formula (1) include an alkyl group. The alkyl group in this case is the same as the above-mentioned alkyl group having 1-10 carbon atoms.

Examples of the di- or higher-valent aromatic hydrocarbon group having 6-30 carbon atoms in the general formula (1) include a di- or higher-valent group formed by removing one or more hydrogen atoms from each of the monovalent groups listed as the aryl group. For example, the divalent group of the aromatic hydrocarbon group having 6-30 carbon atoms is formed by removing one hydrogen atom from each of the monovalent groups listed as the aryl group. The trivalent group of the aromatic hydrocarbon group having 6-30 carbon atoms is formed by removing two hydrogen atoms from each of the monovalent groups listed as the aryl group. The upper limit of the valence of the di- or higher-valent aromatic hydrocarbon group having 6-30 carbon atoms is not particularly limited, but can be tetravalence, for example.

In the general formula (1), “n” is an integer greater than or equal to 2. The upper limit of “n” is not particularly limited, but can be 4, for example.

Specific example of the nitrogen-containing heterocyclic compound represented by the general formula (1) include DPB{1,4-bis(1,10-phenanthroline-2-yl)benzene} represented by the formula (1-1) in the following [Chemical Formula 2], m-DPB represented by the formula (1-2) in the following [Chemical Formula 3], and TPB represented by the formula (1-3) in the following [Chemical Formula 4].

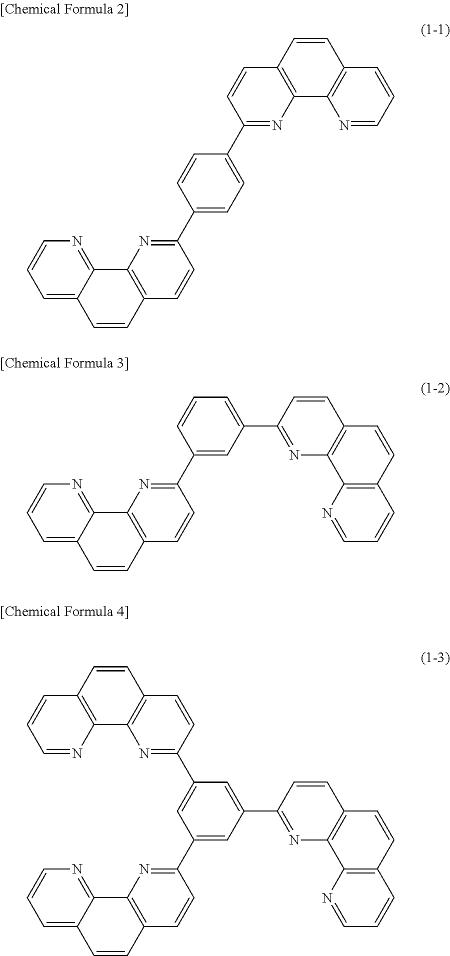 [SMILES: c1cnc2c(c1)ccc1ccc(-c3ccc(-c4ccc5ccc6cccnc6c5n4)cc3)nc12 ; c1cc(-c2ccc3ccc4cccnc4c3n2)cc(-c2ccc3ccc4cccnc4c3n2)c1 ; c1cnc2c(c1)ccc1ccc(-c3cc(-c4ccc5ccc6cccnc6c5n4)cc(-c4ccc5ccc6cccnc6c5n4)c3)nc12]

In the general formula (1), “A” is bonded to a carbon atom at 2-position of 1,10-phenanthroline, but is not limited thereto. “A” may be bonded to any one of carbon atoms at 3- to 9-positions. When “A” is bonded to any one of the carbon atoms at 3- to 9-position, other substituent group (that is, any one of substituent groups of “R¹” to “R⁷”) is not bonded to the carbon atom. Any one of the substituent groups of “R¹” to “R⁷” is bonded to the carbon atom at 2-position. Examples thereof include a compound represented by the general formula (2) in the following [Chemical Formula 5].

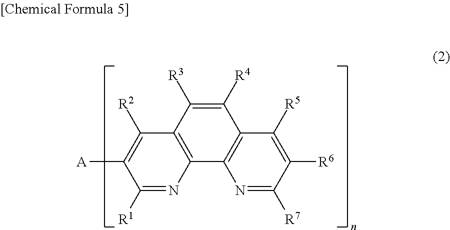

In the nitrogen-containing heterocyclic compound represented by the general formula (2), “A” is bonded to a carbon atom at 3-position of a 1,10-phenanthroline site, and “R¹” is bonded to a carbon atom at 2-position. The other is the same as that of the general formula (1). “R¹” to “R⁷”, “A”, and “n” in the general formula (2) are the same as those of the general formula (1), and the description is omitted herein.

Of course, the nitrogen-containing heterocyclic compound may be a 10-phenanthroline derivative (that is, a 1,10-phenanthroline derivative in which “n” is 1 in the general formula (1)) having only one 1,10-phenanthry group per molecule. Examples of the nitrogen-containing heterocyclic compound include BCP(2,9-dimethyl-4,7-diphenyl-1,10-phenanthroline), Bphen(4,7-diphenyl-1,10-phenanthroline), HNBphen(2-(naphthalen-2-yl)-4,7-diphenyl-1,10-phenanthroline), 2-NPIP(1-methyl-2-(4-(naphthalen-2-yl)phenyl)-1H-imidazo[4,5-f][1,10]phenanthroline), and 1,10-phenanthroline.

Other examples of the nitrogen-containing heterocyclic compound include a 2,2′-bipyridine derivative. For example, a 2,2′-bipyridine derivative having two or more 2,2′-bipyridine sites per molecule can be used.

Examples of the 2,2′-bipyridine derivative include Bpy-OXD(1,3-bis[2-(2,2′-bipyridine-6-yl)-1,3,4-oxadiazo-5-yl]benzene) represented by [Chemical Formula 6], and

Bpy-FOXD(2,7-bis[2-(2,2′-bipyridine-6-yl)-1,3,4-oxadiazo-5-yl]-9,9-dimethylfluorene) represented by [Chemical Formula 7]. These 2,2′-bipyridine derivatives can be said to be a compound having two 2,2′-bipyridyl groups per molecule.

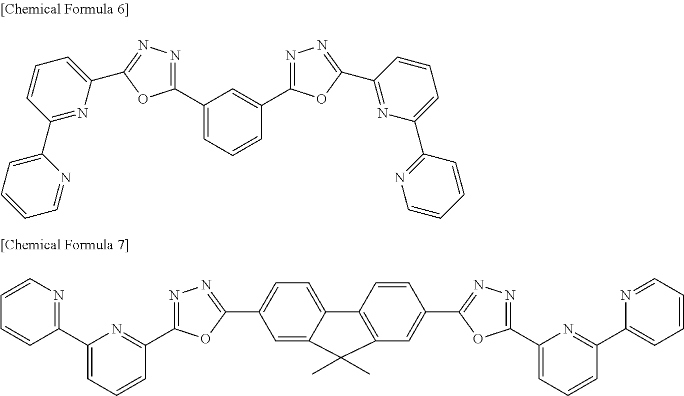 [SMILES: c1ccc(-c2cccc(-c3nnc(-c4cccc(-c5nnc(-c6cccc(-c7ccccn7)n6)o5)c4)o3)n2)nc1 ; CC1(C)c2cc(-c3nnc(-c4cccc(-c5ccccn5)n4)o3)ccc2-c2ccc(-c3nnc(-c4cccc(-c5ccccn5)n4)o3)cc21]

Of course, the nitrogen-containing heterocyclic compound may be a 2,2′-bipyridine derivative having two or more 2,2′-bipyridyl groups per molecule, or may be a 2,2′-bipyridine derivative having only one 2,2′-bipyridyl group. Examples of the nitrogen-containing heterocyclic compound include BP-OXD-Bpy(6,6′-bis[5-(biphenyl-4-yl)-1,3,4-oxadiazo-2-yl]-2,2′-bipyridyl) represented by [Chemical Formula 8], and 2,2′-bipyridine.

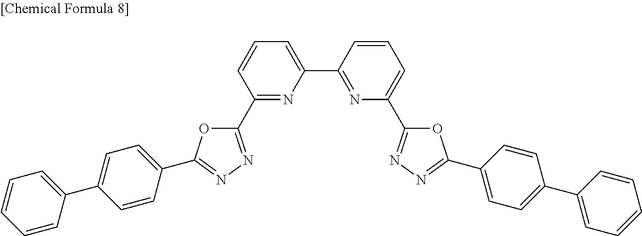 [SMILES: c1ccc(-c2ccc(-c3nnc(-c4cccc(-c5cccc(-c6nnc(-c7ccc(-c8ccccc8)cc7)o6)n5)n4)o3)cc2)cc1]

The nitrogen-containing heterocyclic compound may be a compound having both at least one 1,10-phenanthroline site and at least one 2,2′-bipyridine site, for example, a compound having both at least one 1,10-phenanthry group and at least one 2,2′-bipyridyl group.

The nitrogen-containing heterocyclic compound may have, for example, a 2,9-phenanthroline site, a 3,7-phenanthroline site, and a 3,3′-bipyridine site other than the 1,10-phenanthroline site and the 2,2′-bipyridine site. However, as described later, the nitrogen-containing heterocyclic compound preferably has the 1,10-phenanthroline site and the 2,2′-bipyridine site because the nitrogen-containing heterocyclic compound is easily coordinated to the alkali metal.

Other examples of the nitrogen-containing heterocyclic compound include, but are not limited to, a tris(8-hydroxyquinolinate)aluminum complex (Alq3), TAZ(3-(4-biphenylyl)-4-phenyl-5-(4-tert-buthylphenyl)-1,2,4-triazole), TPBi (2,2′,2″-(1,3,5-benzenetriyl)tris(1-phenyl-1H-benzimidazole), and OXD-7(1,3-bis[5-(p-tert-buthylphenyl)-1,3,4-oxadiazole-2-yl]benzene).

The first layer 3a may contain only the nitrogen-containing heterocyclic compound, or may contain additional material as long as an effect of the present invention to be described later is not inhibited. The additional material may be contained in an amount of 50 mass % or less based on the total mass of materials included in the first layer 3a, for example.

The second layer 3c is made of a material including the nitrogen-containing heterocyclic compound. The second layer 3c is formed so that the second layer 3c is thicker than the alkali metal layer 3b. The second layer 3c preferably has a thickness in a range of 0.2 to 20 nm. The second layer 3c is formed on a surface of the alkali metal layer 3b being closer to the negative electrode 2, i.e., an opposite surface of the alkali metal layer 3b from the surface on which the first layer 3a is formed.

The nitrogen-containing heterocyclic compound contained in the second layer 3c is the same as the nitrogen-containing heterocyclic compounds listed in the description of the first layer 3a, and the description of the nitrogen-containing heterocyclic compound is omitted.

The second layer 3c may be made of only the nitrogen-containing heterocyclic compound, or may contain further material as long as an effect of the present invention to be described later is not inhibited. Materials other than the nitrogen-containing heterocyclic compound and contents of the materials are the same as the additional materials and contents of the additional materials described for the first layer 3a, and thus the description is omitted.

The hole injection layer 3d is made of a material containing an electron-accepting organic material (also referred to as Lewis acid), and is formed on the surface of the second layer 3c being closer to the negative electrode 2.

The electron-accepting organic material is not particularly limited, but may be, for example, made of a pyrazine derivative represented by the structural formula in [Chemical Formula 9].

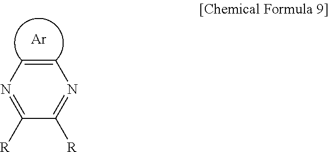

(Herein “Ar” represents an aryl group; and “R” represents hydrogen, an alkyl, alkyloxy or dialkyl amine group having 1-10 carbon atoms, F, Cl, Br, I, or CN.) Furthermore, the electron-accepting substance of the hole injection layer is more preferably a hexaazatriphenylene derivative represented by a structural formula in [Chemical Formula 10].

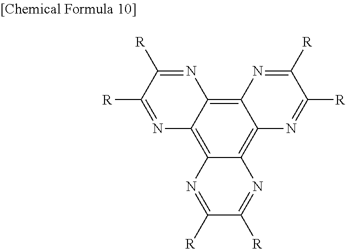

(Herein “R” represents hydrogen, an alkyl, alkyloxy, or dialkyl amine group having 1 to 10 carbon atoms, F, Cl, Br, I, or CN.)

As the hexaazatriphenylene derivative, 1,4,5,8,9,11-hexaazatriphenylene-hexacarbonitrile represented by a structural formula in [Chemical Formula 11] is particularly preferably used. Since electrons provided from hole injection layer 3d can be more efficiently transported to the alkali metal layer 3b in this case, the performance of the organic EL element can be further improved.

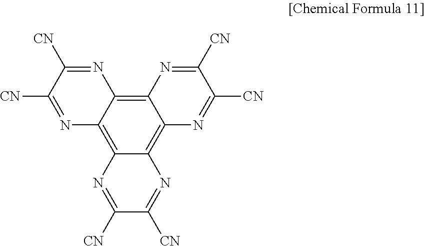

The hole injection layer 3d is preferably made of only the electron-accepting organic material, and may contain additional material as long as an effect of the present invention to be described later is not inhibited.

The thickness of the hole injection layer 3d is not particularly limited, but is preferably in a range of 0.2 to 20 nm. When the thickness is in the range, the hole-injecting efficiency can be properly secured and adjusted.

The process for forming the interlayer 3 constituted by the first layer 3a, the alkali metal layer 3b, the second layer 3c, and the hole injection layer 3d as described above is not particularly limited, but a vacuum deposition method is preferable as it can control a thickness with a high degree of accuracy.

In the organic EL element of the present invention, the interlayer 3 includes the first layer 3a, the alkali metal layer 3b, the second layer 3c, and the hole injection layer 3d as described above. Even if the alkali metal (for example, Li) contained in the alkali metal layer 3b infiltrates the second layer 3c, the interlayer 3 with such a structure allows the nitrogen-containing heterocyclic compound contained in the second layer 3c to catch (trap) the alkali metal. This is because the alkali metal such as Li coordinates to the nitrogen atom in the nitrogen-containing heterocyclic compound. That is, this is because a complex of the nitrogen-containing heterocyclic compound and the alkali metal is formed.

As described above, the alkali metal is trapped by the second layer 3c, which is likely to prevent the alkali metal from diffusing to other layer, for example, the hole injection layer 3d. This suppresses a direct reaction between the alkali metal of the alkali metal layer 3b and the hole injection layer 3d, and mixing of the interface between the alkali metal layer 3b and the hole injection layer 3d, and diffusion of materials between the layers during operation. As a result, it is possible to obtain an organic electroluminescence element having excellent long-term durability and lifetime characteristics.

In addition, since the nitrogen-containing heterocyclic compound is a substance which is likely to coordinate to the alkali metal, the nitrogen-containing heterocyclic compound is likely to trap the alkali metal not only in a room temperature environment but also in a high temperature environment. For this reason, the diffusion preventing function of the second layer 3c is less likely to depend on a temperature environment. Normally, since the diffusibility of the substance increases at a higher temperature, the diffusion preventing function is apt to decrease at the higher temperature. However, in the present embodiment, the diffusion preventing function is less likely to decrease. Therefore, the organic EL element including the interlayer 3 of the present embodiment is likely to prevent the increase in the operation voltage even in the high temperature environment, and has more excellent long-term durability and lifetime characteristics.

Particularly, since the nitrogen-containing heterocyclic compound has higher coordinating performance to the alkali metal when the nitrogen-containing heterocyclic compound has two or more nitrogen atoms per molecule, the effect can be further improved. In the nitrogen-containing heterocyclic compound, the number of nitrogen atoms contained per molecule is particularly preferably 4 or more.

Furthermore, at least two nitrogen atoms in the nitrogen-containing heterocyclic compound are preferably located closer to each other in a molecule. That is, the two nitrogen atoms have a positional relationship in which the nitrogen atoms can coordinate to one alkali metal. In this case, since the nitrogen-containing heterocyclic compound has higher coordinating performance to the alkali metal, the effect can be further improved. Specifically, it is preferable that one heterocycle has two nitrogen atoms as in the above-mentioned 1,10-phenanthroline site, or a plurality of aromatic rings are bonded to each other and each aromatic ring has two nitrogen atoms as in the 2,2′-bipyridine site. The nitrogen-containing heterocyclic compound particularly preferably has two or more (for example, two) 1,10-phenanthroline sites (for example, 1,10-phenanthryl groups) and two or more 2,2′-bipyridine sites (for example, 2,2′-bipyridyl groups) per molecule (for example, the compounds of the formulae (1-1), (1-2), and (1-3)).

In the interlayer 3 of the present embodiment, the alkali metal layer 3b is made of the alkali metal. The alkali metal layer 3b does not contain additional material (for example, an electron-releasing material and an electron transporting organic material). Such a configuration can prevent occurrence of the increase in the operation voltage at the high temperature described above. When the alkali metal layer 3b contains a material other than the alkali metal, the material also may diffuse, and may be insufficiently trapped by the second layer 3c. For this reason, the material is apt to diffuse into the vicinity of the interface between the hole injection layer 3d and the second layer 3c, or the hole injection layer 3d. As a result, a direct reaction of such material with the hole injection layer 3d may cause the increase in the operation voltage. However, when the alkali metal layer 3b is made of the alkali metal as in the present embodiment, the increase in the operation voltage can be suppressed.

Since the second layer 3c is thicker than the alkali metal layer as described above, the alkali metal can be efficiently trapped. The thickness of the second layer 3c is preferably 0.2 to 20 nm, more preferably 0.5 to 5 nm, and particularly preferably 2 to nm.

The thickness of the alkali metal layer 3b is not particularly limited, but is preferably 0.01 to 10 nm, and particularly preferably 0.1 to 5 nm, for prevention of diffusion of the alkali metal and more secure trapping by the second layer 3c.

Since the first layer 3a is also a layer containing the nitrogen-containing heterocyclic compound in the present embodiment, the first layer 3a is likely to prevent the diffusion of the alkali metal of the alkali metal layer 3b as in the second layer 3c. In this case, the first layer 3a can prevent the diffusion of the alkali metal to a layer being closer to the positive electrode 1, and can be less likely to cause the increase in the operation voltage even at the high temperature as in the above.

The thickness of the first layer 3a is not particularly limited, but is preferably 0.5 to 100 nm, and particularly preferably 5 to 100 nm, for prevention of diffusion of the alkali metal and more secure trapping by the first layer 3a.

Herein, the nitrogen-containing heterocyclic compound contained in the first layer 3a is preferably the same as the nitrogen-containing heterocyclic compound contained in the second layer 3c. When a material other than the nitrogen-containing heterocyclic compound is contained in each of the first layer 3a and the second layer 3c, the material contained in the first layer 3a is also preferably the same as the material contained in the second layer 3c. In such a configuration, in the vapor deposition operation of these materials, the number of times of operation required for exchanging the vapor deposition source is reduced. That is, when the nitrogen-containing heterocyclic compound contained in the first layer 3a is different from the nitrogen-containing heterocyclic compound contained in the second layer 3c, the vapor deposition sources should be exchanged in the vapor deposition process. On the other hand, when the nitrogen-containing heterocyclic compound contained in the first layer 3a is the same as the nitrogen-containing heterocyclic compound contained in the second layer 3c, the nitrogen-containing heterocyclic compound can be continuously vapor-deposited, and the alkali metal layer 3b can be vapor-deposited only in a certain region. Therefore, when an in-line filming process by a continuous vapor deposition method described, for example, in JP 2002-348659 A is used, a readily controllable interlayer structure can be formed, which is suited for mass production.

The structure of the nitrogen-containing heterocyclic compound contained in the first layer 3a is the same as the structure of the nitrogen-containing heterocyclic compound contained in the second layer 3c, and thereby the trap amount of the alkali metal being closer to the first layer 3a can be the same as the trap amount of the alkali metal being closer to the second layer 3c. For this reason, the diffusion of the alkali metal to the light emitting unit being closer to the positive electrode 1 and the diffusion of the alkali metal to the light emitting unit being closer to the negative electrode 2 may be almost equivalent to each other, and a decline in a property of one light emitting unit can be likely to be prevented.

Hereinafter, the configurations of components of the organic EL element other than the interlayer 3 will be described.

As shown in FIG. 2, in the organic EL element, a positive electrode 1 is formed on the surface of a substrate 10. A first hole transporting layer 6, a light emitting layer 4 (first light emitting layer 4), a first electron transporting layer 7, the aforementioned interlayer 3, a second hole transporting layer 8, a light emitting layer 5 (second light emitting layer 5), a second electron transporting layer 9, and a negative electrode 2 are stacked in this order on the positive electrode 1. Furthermore, a light-outcoupling layer 12 is formed on an opposite surface of the substrate 10 from the transparent electrode 1. Hereinafter, the present invention will be described based on the present structure as an example, but the structure is merely an example, and the present invention is not limited to the structure but may be applied to other structures unless they go beyond the scope of the present invention.

The substrate 10 may be made of a material having light-transmissvie properties. The substrate 10 may be colorless and transparent, or have a light color. Particularly, in a case of a bottom emission type organic EL element, the substrate 10 is preferably light-transmissive. The substrate 10 may have a frosted glass appearance. Examples of materials for the substrate 10 include transparent glass such as soda-lime glass and alkali-free glass; and plastic such as polyester resin, polyolefin resin, polyamide resin, epoxy resin, and fluorine-based resin. The shape of the substrate 10 may be a film-like shape or a plate-like shape. In addition, the substrate 10 may be a substrate having a light-diffusing effect, which is prepared by adding, into a matrix of the substrate, particles, powders or foams different in refractive index from the matrix or by providing a particular shape to the surface of the substrate. When the light emerges outside without passing through the substrate 10, the substrate 10 may not necessarily be light-transmissive. Any substrate 10 may be used as long as the emission characteristics and lifetime characteristics or the like of the element are not impaired. In particular, a highly heat-conductive substrate 10 may be used in order to suppress the temperature rise of the element by the heat generated during operation.

The positive electrode 1 is an electrode for injecting holes into the organic light emitting layers 4 and 5. The positive electrode 1 is preferably made of an electrode material including a metal, an alloy, an electrically conductive compound which have a higher work function, and a mixture thereof. An electrode material having a work function of 4 eV or more is preferably used. Examples of the materials for the positive electrode 1 include a metal such as gold; CuI, ITO (indium-tin oxide), SnO₂, ZnO, IZO (indium-zinc oxide), a conductive polymer such as PEDOT or polyaniline; a conductive polymer doped with an optional acceptor or the like; and a conductive light-transmitting material such as carbon nanotube. Particularly, in a case of a bottom emission type organic EL element, the positive electrode 1 is preferably light-transmissive.

The positive electrode 1 can be prepared, for example, by forming a thin film made of at least one of these electrode materials on the surface of the substrate 10 by a method such as a vacuum deposition method, a sputtering method, or a coating method. In order to allow light generated in the light emitting layers 4 and 5 to emerge outside through the positive electrode 1, the positive electrode 1 preferably has a light transmittance of 70% or more. Furthermore, the positive electrode 1 preferably has a sheet resistance of several hundreds Ω/□ or less, and particularly preferably 100Ω/□ or less. Herein, the thickness of the positive electrode 1 is preferably equal to or less than 500 nm, and more preferably in a range of 10 to 200 nm, although it depends on the materials used, in order to control the characteristics such as light transmittance and sheet resistance of the positive electrode 1, as described above.

The negative electrode 2 is an electrode for injecting electrons into the light emitting layer. The negative electrode 2 is preferably made of an electrode material including a metal, an alloy, an electrically conductive compound which have a lower work function, and a mixture thereof. An electrode material having a work function of 5 eV or less is preferably used. Examples of the electrode materials for the negative electrode 2 include an alkali metal, an alkali metal halide, an alkali metal oxide, an alkali-earth metal, and an alloy thereof with other metal. Specific examples of the electrode material include sodium, a sodium-potassium alloy, lithium, magnesium, a magnesium-silver mixture, a magnesium-indium mixture, an aluminum-lithium alloy, and an Ai/LiF mixture. Aluminum and an Al/Al₂O₃ mixture or the like can also be used. Furthermore, an alkali metal oxide, an alkali metal halide or a metal oxide may be used for forming a substrate of the negative electrode 2, and one or more layers of conductive materials such as metals may be stacked on the substrate. Examples of such layered structure include an alkali metal/Al layered structure, an alkali metal halide/alkali-earth metal/Al layered structure, and an alkali metal oxide/Al layered structure. A transparent electrode such as ITO or IZO may be used in order to allow light to emerge through the negative electrode 2. The organic matter layer having the interface with the negative electrode 2 may be doped with an alkali or alkali-earth metal such as lithium, sodium, cesium or calcium.

The negative electrode 2 can be prepared, for example, by forming a thin film made of at least one of these electrode materials by a method such as a vacuum deposition method or a sputtering method. To allow light produced in the light emitting layer to emerge through the positive electrode 1, the negative electrode 2 preferably has a light transmittance of 10% or less. On the contrary, when the negative electrode 2 serves as a transparent electrode to allow light to emerge outside through the negative electrode 2 (when light is allowed to emerge through each of the positive electrode 1 and the negative electrode 2), that is, in the case of a top emission type organic EL element, the negative electrode 2 preferably has a light transmittance of 70% or more. The thickness of the negative electrode 2 in the case is normally 500 nm or less, and preferably in a range of 100 to 200 nm, although it depends on the materials used, in order to control the characteristics such as light transmittance of the negative electrode 2.

The material (hole transporting material) included in the first hole transporting layer 6 and the second hole transporting layer 8 is appropriately selected from the group of compounds having a hole transporting property. The material is preferably a compound which has an electron-releasing property or is stable when being subjected to radical cationization due to electron releasing. Examples of the hole transporting materials include a triarylamine-based compound, an amine compound containing a carbazole group, an amine compound containing a fluorene derivative, and starburst amines (m-MTDATA). Representative examples thereof include polyaniline, 4,4′-bis[N-(naphthyl)-N-phenyl-amino]biphenylia-NPD), N,N′-bis(3-methylphenyl)-(1,1′-biphenyl)-4,4′-diamine (TPD), 2-TNATA, 4,4′,4″-tris(N-(3-methylphenyl)N-phenylamino)triphenylamine (MTDATA), 4,4′-N,N′-dicarbazole biphenyl (CBP), spiro-NPD, spiro-TPD, spiro-TAD, and TNB; and 1-TMATA, 2-TNATA, p-PMTDATA, and TFATA as a TDATA-based material. However, the hole transporting materials are not limited to these, and any hole transporting material which is generally known is used. The first hole transporting layer 6 and the second hole transporting layer 8 can be formed by an appropriate method such as a vapor deposition method.

It is preferable that the material (electron transporting material) for forming the first electron transporting layer 7 and the second electron transporting layer 9 is a compound which has the ability to transport electrons, can accept electrons injected from the negative electrode 2, exhibits excellent electron injecting effects on the light emitting layers, prevents the movement of holes to the first electron transporting layer 7 and the second electron transporting layer 9, and has excellent thin film formability. Examples of the electron transporting material include Alq3, an oxadiazole derivative, starburst oxadiazole, a triazole derivative, a phenylquinoxaline derivative, and a silole derivative. Specific examples of the electron transporting material include fluorene, bathophenanthroline, bathocuproine, anthraquinodimethane, diphenoquinone, oxazole, oxadiazole, triazole, imidazole, anthraquinodimethane, 4,4′-N,N′-dicarbazole biphenyl (CBP), and compounds thereof, a metal-complex compound, and a nitrogen-containing five-membered ring derivative. Specific examples of the metal-complex compound include, but are not limited to, tris(8-hydroxyquinolinato)aluminum, tri(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)(o-cresolate)gallium, bis(2-methyl-8-quinolinato)(1-naphtholate)aluminum, and bis(2-methy-8-quinolinato)-4-phenylphenolato. Preferable examples of the nitrogen-containing five-membered ring derivative include oxazole, thiazole, oxadiazole, thiadiazole, and triazole derivatives. Specific examples of the nitrogen-containing five-membered ring derivative include, but are not limited to, 2,5-bis(1-phenyl)-1,3,4-oxazole, 2,5-bis(1-phenyl)-1,3,4-thiazole, 2,5-bis(1-phenyl)-1,3,4-oxadiazole, 2-(4′-tert-butylphenyl)-5-(4″-biphenyl) 1,3,4-oxadiazole, 2,5-bis(1-naphthyl)-1,3,4-oxadiazole, 1,4-bis[2-(5-phenylthiadiazolyl)]benzene, 2,5-bis(1-naphthyl)-1,3,4-triazole, and 3-(4-biphenylyl)-4-phenyl-5-(4-t-butylphenyl)-1,2,4-triazole. Examples of the electron transporting material include a polymer material used for a polymer organic electroluminescence element. Examples of the polymer material include polyparaphenylene and a derivative thereof, and fluorene and a derivative thereof. The thicknesses of the first electron transporting layer 7 and the second electron transporting layer 9 are not particularly limited. For example, the first electron transporting layer 7 and the second electron transporting layer 9 are formed to have a thickness in a range of 10 nm to 300 nm. The first electron transporting layer 7 and the second electron transporting layer 9 can be formed by an appropriate method such as a vapor deposition method.

The light-outcoupling layer 12 can be formed by stacking light-scattering films or microlens films on the opposite surface of the substrate 10 from the positive electrode 1 in order to improve a light diffusion property.

In the embodiment of FIG. 2, the light emitting layer includes a plurality of light emitting layers 4 and 5. The plurality of light emitting layers 4 and 5 are stacked in a direction in which the positive electrode 1 and the negative electrode 2 are stacked, and the interlayer 3 is interposed between the adjacent light emitting layers 4 and 5. As described above, the plurality of light emitting layers 4 and 5 are stacked with the interlayer 3 in-between. The plurality of light-emitting layers 4 and 5 emit light in a state where they are electrically connected in series by the interlayer 3. Hence, it is possible to emit light with high luminance.

Hereinafter, the light emitting layer located closer to the positive electrode 1 than the interlayer 3 is may be referred to as the first light emitting layer 4, and the light emitting layer located closer to the negative electrode 2 than the interlayer 3 is may be referred to as the second light emitting layer 5.

The two light emitting layers 4 and 5 are provided in a state where the interlayer 3 is interposed between the light emitting layers 4 and 5 in the embodiment of FIG. 2.

More additional light emitting layers may be stacked with an interlayer 3 in-between. Although the number of light emitting layers stacked is not particularly limited, the increase in the number of layers causes an increase in complexity in optical and electrical element designs, and thus the number of light emitting layers stacked is preferably 5 or less.

The first light emitting layer 4 and the second light emitting layer 5 may be made of an appropriate electroluminescence material. For example, any of a red light emitting material (wavelength: 605 to 630 nm), a green light emitting material (wavelength: 540 to 560 nm), and a blue light emitting material (wavelength: 440 to 460 nm) may be used. A plurality of light emitting materials may be used.

In the embodiment of FIG. 2, the first light emitting layer 4 includes two layers, i.e., a blue light emitting layer 4a and a green light emitting layer 4b, and the second light emitting layer 5 includes two layers, i.e., a red light emitting layer 5a and a green light emitting layer 5b. For example, the blue light emitting layer 4a and the green light emitting layer 4b can provide fluorescent emission, and the red light emitting layer 5a and the green light emitting layer 5b can provide phosphorescent emission. Thus, light is emitted by using phosphorescence and fluorescence. In particular, emission chromaticity and luminance are adjusted by generating green emission from two kinds of emission, i.e., phosphorescence and fluorescence, and thereby a good emission balance is achieved. The conversion efficiency from electric energy to light can be improved, and changes in luminance and chromaticity can be suppressed even after prolonged emission. That is, the luminance lifetime of green emission is prolonged by stacking two green light emitting layers of green phosphorescence and green fluorescence, and thereby change in chromaticity can be reduced, and lifetime can be prolonged.

Examples of the light emitting material for forming the first light emitting layer 4 and the second light emitting layer 5 include, but are not particularly limited to, perylene (blue), quinacridone (green), Ir(PPy)3 (green), and DCM (red). In addition, any materials known as the materials for organic electroluminescence elements may be used as the materials for the light emitting layer 4. Examples thereof include, but are not limited to, anthracene, naphthalene, pyrene, tetracene, coronene, perylene, phthaloperylene, naphthaloperylene, diphenylbutadiene, tetraphenylbutadiene, coumarin, oxadiazole, bisbenzoxazoline, bisstyryl, cyclopentadiene, quinoline metal complex, tris(8-hydroxyquinolinato)aluminum complex (Alq3), tris(4-methyl-8-quinolinato)aluminum complex, tris(5-phenyl-8-quinolinato)aluminum complex, aminoquinoline metal complex, benzoquinoline metal complex, tri-(p-terphenyl-4-yl)amine, a 1-aryl-2,5-di(2-thienyl)pyrrole derivative, pyran, quinacridone, rubrene, a distyrylbenzene derivative, a distyrylarylene derivative, a distyrylamine derivative, various fluorescent colorants, those materials described above, and the derivatives thereof. It is preferable that light emitting materials selected from these compounds are appropriately mixed. Besides the compounds which emit fluorescent light such as those described above, materials which emit light from spin multiplet such as phosphorescent materials which emit phosphorescent light, and compounds partially containing such a site in the molecule can also be suitably used. The organic layer made of such a material may be formed by a dry process such as vapor deposition or transfer or by a wet process such as spin coating, spray coating, die coating, or gravure printing. The light emitting layers 4 and 5 may be made of the same material or different materials.

The thicknesses of the light emitting layers 4 and 5 are not particularly limited, but are preferably 0.5 to 20 nm.

A method for manufacturing the organic EL element having the above structure is not particularly limited, but can be manufactured by a known manufacturing method.

As described above, the organic EL element of the present invention has the improved interlayer, and is less likely to cause an increase in an operation voltage and short circuit not only in a room temperature environment but also in a high temperature environment. Therefore, the organic EL element is less likely to cause damage due to the increase in the operation voltage, and has excellent long-term durability and lifetime characteristics, as a result. The organic EL element can be widely used in fields such as illuminating light sources, backlights for liquid crystal displays, and flat panel displays.

The organic EL element is available for an illumination device. The illumination device includes the organic EL element. Thereby, the illumination device having high reliability can be obtained. The illumination device may include a plurality of organic EL elements arranged in plane. The illumination device may be a planar illumination body including one organic EL element. The illumination device may have a wiring structure for supplying power to the organic EL element. The illumination device may include a case supporting the organic EL element. The illumination device may include a plug for electrically connecting the organic EL element and a power source to each other. The illumination device can have a panel like structure.

### EXAMPLES

Hereinafter, the present invention will be specifically described with reference to Examples.

### Example 1

There was prepared a glass substrate 10 having a thickness of 0.7 mm. An ITO film having a thickness of 150 nm, a width of 5 mm, and a sheet resistance of about 10Ω/□ was formed as a positive electrode 1 on the glass substrate 10. The substrate 10 was previously washed with a detergent, ion-exchange water, and acetone respectively for 10 minutes under ultrasonication, vapor-washed with IPA (isopropyl alcohol), dried, and then subjected to UV/O₃ treatment.

The substrate 10 was then set in a vacuum evaporator, and a co-vapor deposit of 4,4′-bis[N-(naphthyl)-N-phenyl-amino]biphenyl(α-NPD) and tetrafluoro-tetracyano-quinodimethane (F4-TCNQ) (molar ratio 1:1) having a thickness of 30 nm was formed as a hole injection layer on the surface of the positive electrode 1 formed on the substrate 10 in a reduced-pressure atmosphere at 1×10-4 Pa or less. Next, a film of α-NPD having a thickness of 30 nm was vapor-deposited as a first hole transporting layer 6 on the co-vapor deposit.

Then, a layer of Alq3 and quinacridone (3 mass %) having a thickness of 30 nm was formed as a light emitting layer 4 by co-vapor deposition on the first hole transporting layer 6. A film of pure BCP having a thickness of 60 nm was then formed as a first electron transporting layer 7 on the light emitting layer 4.

An interlayer 3 was prepared in the following manner. First, a film of DPB ([Chemical Formula 2]) represented by the formula (1-1) and having a thickness of 20 nm was formed on the first electron transporting layer 7, to serve a first layer 3a.

Then, a film of Li having a thickness of 0.7 nm was formed on the first layer 3a, to serve an alkali metal layer 3b.

Then, a film of DPB ([Chemical Formula 2]) represented by the formula (1-1) and having a thickness of 3 nm was formed on the alkali metal layer 3b, to serve a second layer 3c.

Furthermore, a film of 1,4,5,8,9,11-hexaazatriphenylene-hexacarbonitrile (HAT-CN6) having a thickness of 10 nm was formed as a hole injection layer 3d on the second layer 3c, to serve the interlayer 3.

A film of α-NPD having a thickness of 40 nm was then vapor-deposited as a second hole transporting layer 8 on the interlayer 3, and a film of Alq3 and quinacridone (7 mass %) having a thickness of 30 nm was formed as a light emitting layer 5 on the second hole transporting layer 8 by co-vapor deposition.

A film of pure BCP having a thickness of 40 nm was then formed as a second electron transporting layer 9 on the light emitting layer 5, and a film of BCP and Li at a molar ratio of 2:1 having a thickness of 20 nm was then formed on the film of pure BCP.

A film of aluminum was then vapor-deposited as a negative electrode 2 at a vapor deposition rate of 0.4 nm/s in a width of 5 mm and a thickness of 100 nm.

In this way, there was obtained an organic EL element including the two light emitting layers 4 and 5 and the interlayer 3 provided between the light emitting layers 4 and 5.

### Example 2

An organic EL element was obtained in the same manner as in Example 1 except that a first layer 3a and a second layer 3c were made of BCP (nitrogen-containing heterocyclic compound) in place of DPB.

### Example 3

An organic EL element was obtained in the same manner as in Example 1 except that a first layer 3a and a second layer 3c were made of Bphen (nitrogen-containing heterocyclic compound) in place of DPB.

### Example 4

An organic EL element was obtained in the same manner as in Example 1 except that a first layer 3a and a second layer 3c were made of Alq3 (nitrogen-containing heterocyclic compound) in place of DPB.

### Example 5

An organic EL element was obtained in the same manner as in Example 1 except that a second layer 3c was made of BCP (nitrogen-containing heterocyclic compound) in place of DPB.

### Example 6

An organic EL element was obtained in the same manner as in Example 1 except that a first layer 3a and a second layer 3c were made of m-DPB ([Chemical Formula 3]) represented by the formula (1-2) in place of DPB.

### Example 7

An organic EL element was obtained in the same manner as in Example 1 except that a second layer 3c was made of m-DPB ([Chemical Formula 3]) represented by the formula (1-2) in place of DPB.

### Example 8

An organic EL element was obtained in the same manner as in Example 1 except that an alkali metal layer 3b was made of Na in place of Li.

### Example 9

An organic EL element was obtained in the same manner as in Example 1 except that the thickness of a second layer 3c was 25 nm.

### Comparative Example 1

An organic EL element was obtained in the same manner as in Example 1 except that an alkali metal layer 3b was made of Li₂O in place of Li.

### Comparative Example 2

An organic EL element was obtained in the same manner as in Example 1 except that a second layer 3c was not formed.

### Comparative Example 3

An organic EL element was obtained in the same manner as in Comparative Example 3 except that a second layer 3c was not formed.

### Comparative Example 4

An organic EL element was obtained in the same manner as in Example 1 except that a first layer 3a and a second layer 3c were made of a compound (compound containing no nitrogen atom other than a nitrogen-containing heterocyclic compound) represented by [Chemical Formula 12] in place of DPB.

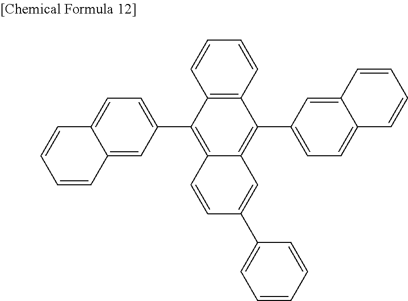 [SMILES: c1ccc(-c2ccc3c(-c4ccc5ccccc5c4)c4ccccc4c(-c4ccc5ccccc5c4)c3c2)cc1]

### Comparative Example 5

An organic EL element was obtained in the same manner as in Example 1 except that an alkali metal layer 3b was made of Li₂WO₄ in place of Li.

### Comparative Example 6

An organic EL element was obtained in the same manner as in Example 1 except that an alkali metal layer 3b was a stack of layers of Li and DPB (at a thickness ratio of 10:90) in place of Li.

There were measured elevated values ▪T of operation voltages when a current of 4 mA/cm² was applied to each of the organic EL elements obtained in Examples and Comparative Examples at 30° C. and 80° C. (▪T=(operation voltage after current test for 300 hours)−(operation voltage in early stage of current test (0 hour)). The results are shown in Table 1. Materials used in the first layer 3a, the alkali metal layer 3b, and the second layer 3c are also collectively shown in Table 1.

TABLE 1

| | | | | Increase | Increase |
|---|---|---|---|---|---|
| | | | | in voltage | in voltage |
| | | | | (V) after | (V) after |
| | | | | current is | current was |
| Examples/ | | Alkali | | applied at | applied at |
| Comparative | First | metal | Second | 30° C. for | 80° C. for |
| Examples | layer 3a | layer 3b | layer 3c | 300 hours | 300 hours |
| Example 1 | DPB | Li | DPB | 0.01 | 0.1 |
| Example 2 | BCP | Li | BCP | 0.02 | 0.22 |
| Example 3 | Bphen | Li | Bphen | 0.02 | 0.21 |
| Example 4 | Alq3 | Li | Alq3 | 0.02 | 0.23 |
| Example 5 | DPB | Li | BCP | 0.03 | 0.28 |
| Example 6 | m-DPB | Li | m-DPB | 0.02 | 0.14 |
| Example 7 | DPB | Li | m-DPB | 0.02 | 0.17 |
| Example 8 | DPB | Na | DPB | 0.01 | 0.1 |
| Example 9 | DPB | Li | DPB | 0.01 | 0.1 |
| Comparative | DPB | Li₂O | DPB | 0.2 | 2.03 |
| Example 1 | | | | | |
| Comparative | DPB | Li | | 0.33 | 3.35 |
| Example 2 | | | | | |
| Comparative | DPB | Li₂O | | 0.47 | 4.53 |
| Example 3 | | | | | |
| Comparative | Non-N | Li | Non-N | 0.17 | 1.89 |
| Example 4 | | | | | |
| Comparative | DPB | Li₂WO₄ | DPB | 0.05 | 0.89 |
| Example 5 | | | | | |
| Comparative | DPB | Li:DPB | DPB | 0.15 | 1.33 |
| Example 6 | | | | | |

From the results of Table 1, it is apparent that the increase in the operation voltage in a high temperature environment can be suppressed when the alkali metal layer 3b is made of the alkali metal, and the first layer 3a and/or the second layer 3c are/is made of the nitrogen-containing heterocyclic compound. It is also found that the increase in the operation voltage in the high temperature environment is further suppressed when the first layer 3a or the second layer 3c is made of the nitrogen-containing heterocyclic compound represented by the general formula (1). In particular, the increase in the operation voltage in the high temperature environment is less than 0.2 V in Examples 1, 6, 7, 8, and 9. From these results, it is found that the increase in the operation voltage in the high temperature environment is particularly preferably suppressed when both the first layer 3a and the second layer 3c are made of the nitrogen-containing heterocyclic compound represented by the general formula (1).

As described above, it is found that Example 9 provides the same result as that in Example 1, can suppress an increase in a voltage during operation, and particularly effectively suppresses the increase in the operation voltage in the high temperature environment. However, since the thickness of the second layer 3c was more than 20 nm in Example 9, both the absolute values of the operation voltages when a current of 4 mA/cm² was applied at 30° C. and 80° C. were increased by about 3 V as compared with Example 1.

On the other hand, in Comparative Examples 1, 3 5, and 6, when the alkali metal layer 3b was made of a metal oxide, or a mixed material containing an alkali metal and a material other than the alkali metal even if the first layer 3a and the second layer 3c were made of the nitrogen-containing heterocyclic compound, the operation voltage was remarkably increased at both a normal temperature and a high temperature. Since the second layer 3c was not provided in Comparative Example 2, the operation voltage was remarkably increased at both the normal temperature and the high temperature.

### REFERENCE SIGNS LIST

1 Positive electrode
2 Negative electrode
3 Interlayer
3a First layer
3b Alkali metal layer
3c Second layer
3d Hole injection layer
4 Light emitting layer (first light emitting layer)
5 Light emitting layer (second light emitting layer)

1. An organic electroluminescence element comprising:
a positive electrode;
a negative electrode;
a plurality of light emitting layers interposed between the positive electrode and the negative electrode; and
an interlayer provided between two adjacent light emitting layers of the plurality of light emitting layers,
the interlayer including:
a first layer containing a nitrogen-containing heterocyclic compound;
an alkali metal layer containing an alkali metal;
a second layer containing a nitrogen-containing heterocyclic compound; and
a hole injection layer containing an electron-accepting organic material,
the first layer, the alkali metal layer, the second layer, and the hole injection, and layer being arranged in this order from the positive electrode to the negative electrode.

2. The organic electroluminescence element according to claim 1, wherein
the second layer has a thickness in a range of 0.2 to 20 nm.

3. The organic electroluminescence element according to claim 1, wherein
the nitrogen-containing heterocyclic compound has two or more 1,10-phenanthroline sites or two or more 2,2′-bipyridine sites per molecule.

4. The organic electroluminescence element according to claim 1, wherein
the nitrogen-containing heterocyclic compound contained in the first layer is the same as the nitrogen-containing heterocyclic compound contained in the second layer.

5. The organic electroluminescence element according to claim 1, wherein
the electron-accepting organic material is 1,4,5,8,9,11-hexaazatriphenylene-hexacarbonitrile.

6. An illumination device comprising the organic electroluminescence element according to claim 1.

7. The organic electroluminescence element according to claim 1, wherein the second layer being thicker than the alkali metal layer.

8. An illumination device comprising the organic electroluminescence element according to claim 7.
